(19) United States
(12) Patent Application Publication
(10) Pub. No.: US 2002/0034802 A1
(43) Pub. Date: Mar. 21, 2002

(54) Crystals of the alpha 1 beta 1 integrin I-domain and their use

(76) Inventors: Michael Karpusas, Roslindale, MA (US); Matthias Nolte, Salem, NH (US)

Correspondence Address: Niki D. Cox, Biogen, Inc., 14 Cambridge Center, Cambridge, MA, 02142, US

(21) Appl. No.: 09/826,716

(22) Filed: Apr. 5, 2001

Related U.S. Application Data
(63) Continuation of application No. PCT/US99/23261, filed on Oct. 6, 1999.
(60) Provisional application No. 60/103,301, filed on Oct. 6, 1998.

(51) Int. Cl. C12N 9/00

(52) U.S. Cl. 435/183; 23/300

(57) ABSTRACT

The present invention relates to crystals of fragments of alpha 1 beta 1 integrin {“α1β1”), specifically, a soluble fragment of the α1 chain of α1β1 integrin (143-340). The invention relates further to uses of these crystals and the coordinates thereof to design, identify, optimize or characterize chemical entities having properties of interest.

### RELATED APPLICATIONS

This is a continuation of PCT/US99/23261, filed on Oct. 6, 1999 as a continuation of prior U.S. provisional Ser. No. 60/103,301, filed Oct. 6, 1998. The entire disclosure of each of the aforesaid patent applications are incorporated herein by reference.

### BACKGROUND OF THE INVENTION

A major class of cell receptors that interacts with the constituents of the extracellular matrix (“ECM”) (e.g., collagen, laminin) are the integrins which are transmembrane heterodimeric glycoproteins composed of noncovalently associated a and β subunits. The integrin family contains at least 16α subunits, seven of which contain an approximately 200 amino acid inserted domain in their N-terminal region variously called the “I-domain” or the “A-domain”.

Processes such as cell differentiation, cell proliferation and cell migration in embryonic development, as well as remodeling and cell/tissue repair events, are dependent on communication of cells with the ECM. Alpha 1 beta 1 integrin (“α1β1 integrin”) is a cell-surface receptor for collagen I, collagen IV and laminin. It is also known as VLA- 1. Indeed, α1β1 supports not only collagen-dependent adhesion and migration, but also is likely to be a critical collagen receptor on mesenchymally-derived cells that may be involved in ECM remodeling after injury (Gotwals et al.(1996), J. Clin. Invest. 97: p 2469-2477 ). The ability of cells to contract and organize collagen matrices is a critical component of any wound healing response. Improper regulation of α1β1 integrin may result in certain pathological conditions such as fibrosis.

Moreover, there is a limited, but provocative, literature suggesting that α1β1 may play a role in T cell/monocyte driven diseases. Anti-α1β1 antibodies block T-cell dependent cytokine expression. Miyake et al., J. Exp. Med., 177: 863-868 (1993). Expression of α1β1 is upregulated in persistently activated, 2-4 week old cultured T cells (Hemler et al., Eur. J. Immunol., 15: 502-508 (1985)) and is also expressed on a high percentage of T cells isolated from the synovium of patients with rheumatoid arthritis. Hemiler et al., J. Clin. Invest., 78: 696-702 (1986). Chronic tissue damage results from both resident activated T cells, and also monocytes/fibroblasts recruited by T cell-derived cytokines. Blocking the α1β1-induced T cell interaction might relieve tissue damage by removing activated T cells and/or by diminishing inflammatory cytokine levels.

It would therefore be useful to design, identify or obtain potential drug candidates which would interfere with the α1β1 integrin-ECM or T-cell interaction(s). The recent emergence of drug design to identify candidates that play a role in a physiologically relevant biological pathway has provided a useful approach for obtaining, or designing, lead compounds for drugs.

Generally, this approach requires selecting a protein target molecule which plays a role in a physiologically relevant biological pathway. Typically, once an inhibitor or agonist, natural or synthesized, is found for the target molecule, it is modified or optimized to produce a candidate with the desired properties.

In order to more efficiently design or modify a ligand, it is useful to have a three-dimensional structure for the bioactive conformation of a known ligand as it binds to the target protein molecule. Furthermore, it is valuable to understand the detailed interactions of the ligand with its target protein by examining the three-dimensional structure of the protein target in complex with its known ligand. This allows the artisan to preserve the critical interactions with the protein, while modifying candidate ligands to interact more precisely with the protein, resulting in better potency and specificity.

However, the three dimensional crystal structure of the protein target is frequently unavailable due to the significant effort required to obtain crystals of sufficient size and quality to provide accurate information regarding the structure. For example, it is time consuming and often difficult to express, purify and characterize a protein. Additionally, once the protein of sufficient purity is obtained, it must be crystallized to a size and quality which is useful for x-ray diffraction and subsequent structure solution. Thus, although crystal structures can provide a wealth of valuable information in the field of drug design and discovery, crystals of certain biologically relevant molecules such as α1β1 integrin, are not readily available to those skilled in the art.

Furthermore, although the amino acid sequence of a target protein, such as α1β1 integrin, is known, this sequence information does not allow an accurate prediction of the crystal structure of the protein. Nor does the sequence information afford an understanding of the structural, conformational and chemical interactions between a ligand such as α1β1 integrin and its target.

Thus, there is a need for a detailed knowledge of the crystalline three-dimensional structure of the extracellular domain of α1β1 integrin, to effectively design, screen or optimize compounds capable of interfering with the α1β1 integrin-ECM and/or T-cell interactions.

A soluble version of α1β1 integrin can be made from its extracellular region or fragments thereof. As used herein, the term “α1β1 integrin” includes soluble α1β1 integrin polypeptides lacking transmembrane and intracellular regions, homologs and analogs of α1β1 integrin or derivatives thereof. Crystals of the α1 chain of a α1β1 integrin or fragments thereof of a size and quality such as described herein, would allow performance of x-ray diffraction studies and enable those skilled in the art to conduct studies relating to the binding properties of α1β1 integrin, as well as the binding properties of molecules or molecular complexes which may associate with α1β1 integrin or fragments thereof.

### SUMMARY OF THE INVENTION

Accordingly, the present invention is directed to crystals of the α1 chain of α1β1 integrin or crystals of fragments of the α1 chain, of sufficient size and quality to obtain useful information about the properties of α1β1 integrin and molecules or complexes which may associate with it. The claimed invention provides the three-dimensional crystal structure of the Cys 143 to Ala340 fragment of the α1 chain of α1β1 integrin, which can be used to identify binding sites to solve the structure of unknown crystals, to provide mutants having desirable binding properties, and ultimately, to design, characterize, or identify molecules or chemical entities capable of interfering with the interaction between collagen or other ligands and α1β1.

Additional features and advantages of the invention will be set forth in the description which follows, and in part will be apparent from the description, or may be learned by practice of the invention. The objectives and other advantages of the invention will be realized and attained by the compositions and methods particularly pointed out in the written description and claims hereof, as well as in the appended drawings.

To achieve these and other advantages, and in accordance with the purpose of the invention, as embodied and broadly described herein, the invention relates to a crystal of α1β1 integrin. More particularly, the invention relates to a crystal formed by a functional fragment of the extracellular domain of the α1 chain of α1β1 (Cys 143-Ala340), wherein the crystal has cell constants a=34.77 Å, b=85.92 Å, c=132.56 Å, α=β=γ=90 Å, and a space group of P2₁2₁2₁, and equivalents of that crystal. The claimed crystals of α1β1 are substantially described by the structural coordinates identified in Table II. The claimed crystals in certain embodiments are characterized by a binding site moiety comprising Asp154, Ser156, Asn157,Ser158, Leu222, Gln223, Thr224, Asp257, Glu259, His261, His288, Tyr289, Gly292, Leu294 and Lys298. Mutants, homologs, co-complexes and fragments of the claimed crystals are also contemplated herein.

The claimed invention in certain embodiments relates to heavy atom derivatives of the crystallized form of α1β1 integrin (143-340), and, more specifically, the heavy atom derivatives of the crystallized form of α1β1 described above. In various embodiments, the claimed invention relates to methods of preparing crystalline forms of α1β1, or fragments thereof, by providing an aqueous solution comprising at least a fragment of α1β1, providing a reservoir solution comprising a precipitating agent, mixing a volume of the α1β1 solution with a volume of the reservoir solution and crystallizing the resultant mixed volume. In certain embodiments, the crystal is derived from an aqueous solution comprising the α1 chain of α1β1 (Cys143-Ala340). In various embodiments, the concentration of α1β1 in the aqueous solution is about 1 to about 50 mg/ml, preferably about 5 mg/ml to about 15 mg/ml, and most preferably, about 10 mg/ml. The precipitating agents used in the invention may be any precipitating agent known in the art, preferably one selected from the group consisting of sodium citrate, ammonium sulfate and polyethylene glycol. Any concentration of precipitating agent may be used in the reservoir solution, however it is preferred that the concentration be about 20% weight per volume (“w/v”) to about 50% w/v, more preferably about 25% w/v. Similarly, the pH of the reservoir solution may be varied, preferably between about 4 to about 10, most preferably about 6.5.

Various methods of crystallization can be used in the claimed invention, including, but not limited to, vapor diffusion, batch, liquid bridge, or dialysis. Vapor diffusion crystallization is preferred.

Additionally, the claimed invention relates to methods of using the claimed crystal, and the structural coordinates, in methods for screening, designing, or optimizing molecules or other chemical entities that may interfere with the interaction between α1β1 ligands such as members of the extracellular matrix (e.g., collagen) and α1β1. Thus, the structural coordinates of α1β1 or portions thereof can be used to solve the crystal structure of a mutant, homologue or co-complex of α1β1 or a fragment thereof, as well as to solve other unknown crystals which associate with α1β1 or fragments thereof.

In some embodiments, the structural coordinates of the α1 chain of α1β1 (as exemplified in Table II) can be used to evaluate a chemical entity to obtain information about the binding of the chemical entity to α1β1. The structural coordinates can be used to characterize chemical entities which interfere with the relationship between the extracellular matrix (i.e., collagen or laminin) and α1β1 such as inhibitors or agonists. The coordinates can also be used to optimize binding characteristics, to determine the orientation of ligands in a binding site of α1β1. One skilled in the art will appreciate the numerous uses of the claimed invention in the areas of drug design, screening and optimization of drug candidates, as well as in determining additional unknown crystal structures.

In various embodiments, the claimed invention relates to a machine readable data storage medium having a data storage material encoded with machine readable data, which, when read by an appropriate machine, can display a three dimensional representation of a crystal. The crystals displayed comprise a fragment of α1β1 such as that described by the coordinates in Table II, or a crystal having a binding site moiety comprising amino acids Asp154, Ser156, Asn157,Leu222, Gln223, Thr224, Asp257, Glu259, His261, His288, Tyr289, Gly292, Leu294 and Lys298.

In other embodiments, the claimed invention relates to a method for determining a at least a portion of a three dimensional structure of a chemical entity or molecular complex by calculating phases from the structural coordinates of a crystal of a fragment of α1β1 calculating the electron density map from the phases obtained, and then determining at least a portion of the unknown structure based upon the electron density map.

In yet other embodiments, the invention relates to methods for evaluating the ability of a chemical entity to associate with α1β1. The methods employ computational or experimental means to perform a fitting operation between the chemical entity and the α1β1 to obtain data related to the association, and analyzing the data to determine the characteristics. Chemical entities identified by these methods which are capable of interfering with the in vivo or in vitro association between the extracellular matrix and α1β1 are also encompassed by the claimed invention. The claimed chemical entities may comprise binding sites substantially similar to those of α1β1, or, alternatively may comprise binding sites capable of associating with the binding sites of α1β1.

It is to be understood that both the foregoing general description and the following detailed description are exemplary and explanatory and are intended to provide further explanation of the invention as claimed.

The accompanying drawings are included to provide a further understanding of the invention and are incorporated in and constitute a part of this specification, illustrate several embodiments of the invention, and together with the description, serve to explain the principles of the invention.

### BRIEF DESCRIPTION OF THE FIGURES

FIG. 1: 2 Fo-Fc electron density map for a representative region of the α1 I-domain crystal structure, contoured at 1 Sigma.

FIG. 2: Ribbon representation of the fold of the α1 I-domain molecule. The arrow points to the MIDAS binding site.

### DETAILED DESCRIPTION OF THE INVENTION

In order that the invention described herein may be more fully understood, the following detailed description is set forth.

The present invention relates to a crystal of a soluble fragment of the extracellular domain of the α1β1 integrin. Specifically, it relates to a crystal of a soluble protein comprising the sequence from Cys143 to Ala340 of the α1 chain of α1β1 integrin (“sα1β1(143-340)”), the structure of sα1β1(143-340) as determined by X-ray crystallography, and the use of the sα1β1(143-340) structure and that of its homologs, mutants and co-complexes to design, identify, characterize, screen and/or optimize candidate inhibitors or agonists of α1β1 activity.

A. DEFINITIONS

The term α1β1 integrin (“VLA-1” or “α1β1” or “α1β1 integrin”, used interchangeably) herein refers to a genus of polypeptides which are capable of binding to members of the extracellular matrix proteins such as laminin or collagen, or homologs or fragments thereof. The term as used herein includes sα1β1 integrin 143-340), homologs, mutants, equivalents and fragments thereof.

The term “co-complex” refers to an α1β1 or a mutant or homolog of α1β1 in covalent or non-covalent association with a chemical entity.

The term “homolog” or “homologous”—as used herein is synonymous with the term “identity” and refers to the sequence similarity between two polypeptides, molecules or between two nucleic acids. When a position in both of the two compared sequences is occupied by the same base or amino acid monomer subunit (for instance, if a position in each of the two DNA molecules is occupied by adenine, or a position in each of two polypeptides is occupied by a lysine), then the respective molecules are homologous at that position. The percentage homology between two sequences is a function of the number of matching or homologous positions shared by the two sequences divided by the number of positions compared×100. For instance, if 6 of 10 of the positions in two sequences are matched or are homologous, then the two sequences are 60% homologous. By way of example, the DNA sequences CTGACT and CAGGTT share 50% homology (3 of the 6 total positions are matched). Generally, a comparison is made when two sequences are aligned to give maximum homology. Such alignment can be provided using, for instance, the method of Needleman et al., J. Mol Biol. 48: 443-453 (1970), implemented conveniently by computer programs such as the Align program (DNAstar, Inc.). Homologous sequences share identical or similar amino acid residues, where similar residues are conservative substitutions for, or “allowed point mutations” of, corresponding amino acid residues in an aligned reference sequence. In this regard, a “conservative substitution” of a residue in a reference sequence are those substitutions that are physically or functionally similar to the corresponding reference residues, e.g., that have a similar size, shape, electric charge, chemical properties, including the ability to form covalent or hydrogen bonds, or the like. Particularly preferred conservative substitutions are those fulfilling the criteria defined for an “accepted point mutation” in Dayhoff et al., 5: Atlas of Protein Sequence and Structure, 5: Suppl. 3, chapter 22: 354-352, Nat. Biomed. Res. Foundation, Washington, D.C. (1978).

The term “mutant” refers to an α1β1 integrin or fragment thereof, characterized by the replacement, deletion, or insertion of at least one amino acid from the wild-type. Such a mutant may be prepared, for example, by expression of α1β1 integrin previously altered in its coding sequence by oligonucleotide-directed mutagenesis.

The term “positively charged amino acid” includes any amino acid, natural or unnatural, having a positively charged side chain under normal physiological conditions. Examples of positively charged naturally occurring amino acids are arginine, lysine and histidine.

The term “negatively charged amino acid” includes any amino acid, natural or unnatural, having a negatively charged side chain under normal physiological conditions. Examples of negatively charged naturally occurring amino acids are aspartic acid and glutamic acid.

The term “hydrophobic amino acid” means any amino acid having an uncharged, nonpolar side chain that is relatively insoluble in water. Examples are alanine, leucine, isoleucine, valine, proline, phenylalanine, tryptophane and methionine.

The term “hydrophilic amino acid” means any amino acid having an uncharged, polar side chain that is relatively soluble in water. Examples are serine, threonine, tyrosine, asparagine, glutamine, and cysteine.

The term “altered surface charge” means a change in one or more of the charge units of a mutant polypeptide, at physiological pH, as compared to α1β1 integrin. The change in surface charge can be determined by measuring the isoelectric point (pI) of the polypeptide molecule containing the substituted amino acid and comparing it to the pH of the wild-type molecule.

The term “associating with” refers to a condition of proximity between two chemical entities, or portions thereof, for example, an α1β1 integrin or portions thereof and a chemical entity. The association may be non-covalent, wherein the juxtaposition is energetically favored by hydrogen bonding, van der Waals interaction, or electrostatic interaction, or it may be a covalent association.

The term “binding site” refers to any or all of the sites where a chemical entity binds or associates with another entity.

The term “structural coordinates” refers to the coordinates derived from mathematical equations related to the patterns obtained on diffraction of a monochromatic beam of X-rays by the atoms (scattering centers) of molecule in crystal form. The diffraction data are used to calculate an electron density map of the repeating units of the crystal. Those skilled in the art will understand that the data obtained are dependent upon the particular system used, and hence, different coordinates may in fact describe the same crystal if such coordinates define substantially the same relationship as those described herein. The electron density maps are used to establish the positions of the individual atoms within the unit cell of the crystal.

Those of skill in the art understand that a set of structural coordinates determined by X-ray crystallography is not without standard error. Table II is the atomic coordinates of the I-domain of the α1 chain of α1β1 integrin (143-340). For the purpose of this invention, any set of structural coordinates of α1β1 (143-340) that have a root mean square deviation of equivalent protein backbone atoms of less than about 2 Å when superimposed—using backbone atoms—on the structural coordinates in Table II shall be considered identical. Preferably the deviation is less than about 1 Å and more preferably less than about 0.5 Å.

The term “heavy atom derivatization” refers to a method of producing a chemically modified form of a crystallized α1β1 integrin. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location of the bound heavy metal atom(s) can be determined by X-ray diffraction analysis of the soaked crystal. This information can be used to generate the phase information used to construct the three-dimensional structure of the molecule.

The term “unit cell” refers to a basic shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “molecular replacement” refers to a method that involves generating a preliminary structural model of a crystal whose structural coordinates are unknown, by orienting and positioning a molecule whose structural coordinates are known e.g. the α1β1 I-domain coordinates in Table II, within the unit cell of the unknown crystal, so as to best account for the observed diffraction pattern of the unknown crystal. Phases can then be calculated from this model, and combined with the observed amplitudes to give an approximate Fourier synthesis of the structure whose coordinates are unknown. This in turn can be subject to any of the several forms of refinement to provide a final accurate structure of the unknown crystal. See, e.g., Lattman, E., “Use of the Rotation and Translation Functions”, Methods in Enzymology, 115, pp. 55-77 (1985); Rossman, ed., “The Molecular Replacement Method”, Int. Sci. Rev. Ser. No. 13, Gordon and Breach, New York (1972), all specifically incorporated by reference herein. Using the structural coordinates provided by this invention, molecular replacement may be used to determine the structural coordinates of a crystalline co-complex, unknown ligand, mutant, homolog, or of a different crystalline form of α1β1 or fragment thereof. Additionally, the claimed crystal and its coordinates may be used to determine the structural coordinates of a chemical entity which associates with α1β1 or fragment or with a member of the extracellular matrix which is a ligand for α1β1 or fragment thereof.

The term “chemical entity” as used herein shall mean, for example, any molecule, molecular complex, compound or fragment thereof.

Mutants of α1β1 or fragments thereof may be generated by site-specific incorporation of natural or unnatural amino acids into α1β1 or fragments using general biosynthetic methods known to those skilled in the art. For example, the codon encoding the amino acid of interest in wild-type α1 chain of α1β1 may be replaced by a “blank” nonsense codon, such as TAG, using oligonucleotide-directed mutagenesis. A suppressor tRNA directed against this codon can then be chemically aminoacylated in vitro with the desired amino acid. The aminoacylated tRNA can then be added to an in vitro translation system to yield a mutant α1β1 with the site-specific incorporated amino acid.

The term “soluble fragment” of α1β1 and any equivalent term used herein, refers to a functional fragment of α1β1, and more particularly refers to a functional α1 chain. The term “functional” as used in this context refers to a soluble fragment of the extracellular domain that is capable of binding to, or associating with a member of the extracellular matrix such as collagen or laminin or any fragments or homologs thereof, including molecular complexes comprising fragments thereof. Such binding may be demonstrated through immunoprecipitation experiments, using standard protocols known in the art.

A. ALPHA 1 BETA 1 INTEGRIN, its Crystal, and its Biological Implications

It will be understood that throughout the specification and claims, the positional location of the amino acids described is not an absolute value, but rather, defines the relative relationship of the residues. Thus it is intended that the present invention encompass the sequences having the same or similar relative positions.

For the first time, the present invention permits the use of molecular design techniques to design, screen and optimize chemical entities and compounds, including inhibitory compounds, capable of binding to the active site or accessory binding site of α1β1, in whole or in part. The α1β1 integrin is a membrane-bound protein of considerable biomedical interest because of its involvement in important functions mediated by its binding to the extracellular matrix such as collagen. Since α1β1 is found in various vertebrate (e.g., mammalian) organisms, such as humans, mice, rats, and pigs, the claimed invention is not intended to be limited to any particular species or organism.

The α1β1 integrin (VLA- 1)is a member of the integrin family of proteins. The crystal structure of I-domains from other members of this family, αM, αL and α2, have been described. See Dickeson & Santoro (1998) Cell. Mol. Life Sci. 54, 556-566 for a review and Emsley et al., J. Biol. Chem. 272, 28512-28517.

These I-domains were used as a framework for understanding the sα1β1 integrin (143-340) crystal structure. However, despite certain similarities, the differences between the I-domain of α1 and the I-domains of αM, αL, and α2 integrins, confirm that these ligand-receptor systems utilize spatially overlapping, but nonidentical and nonconserved sites of contact residues with different molecular determinants of binding.

Considering the complexity and overlap of the various integrins and their biological processes, the fact that α1β1 binds specifically to its ligand suggests that inhibiting α1β1 signaling may have important therapeutic applications. The crystal structure of sα1β1 (143-340) presented here is expected to be useful in the design, identification, characterization and optimization of such therapeutic agents.

The following detailed description of applicants invention encompasses the (a) crystal structure of the α1 chain I-domain (Cys143-Ala340) of α1β1 integrin and the coordinates thereof, (b) the binding sites thereof, (c) methods of making an α1β1 crystal or fragment thereof, and (d) methods of using the α1β1 crystal or fragment thereof and its structural coordinates.

### (a) Crystal Structure of the α1 I-domain

The claimed invention provides crystals of α1β1 integrin as well as the structure derived therefrom. The crystals are derived from the α1 I-domain of the rat. Nevertheless, the sequence identity between rat and human alpha 1 I-domains is about 95%. Specifically, the amino acids which differ between the rat and human α1 I-domains are Ile166, Asn214, Gly217, Arg 218, Gln219 Leu222, Tyr262, Gln267, His288, Ala330 (rat I-domain sequence). Most of them are located a relatively long distance away from the metal-ion-dependent-adhesion-site (MIDAS) of the α1 I-domain, the site likely to be involved in ligand binding. The only 2 amino acids that are expected to participate in ligand binding are the Leu222 and His288. This high degree of primary amino acid sequence identity indicates that the 3-dimensional structures of rat and human 1 I-domains are expected to be similar. Therefore, we used the crystal structure of the rat 1 I-domain for the purposes discussed in this patent and we fully expect that the 3-dimensional structure of the human 1 I-domain will have substantially identical coordinates for the main chain atoms.

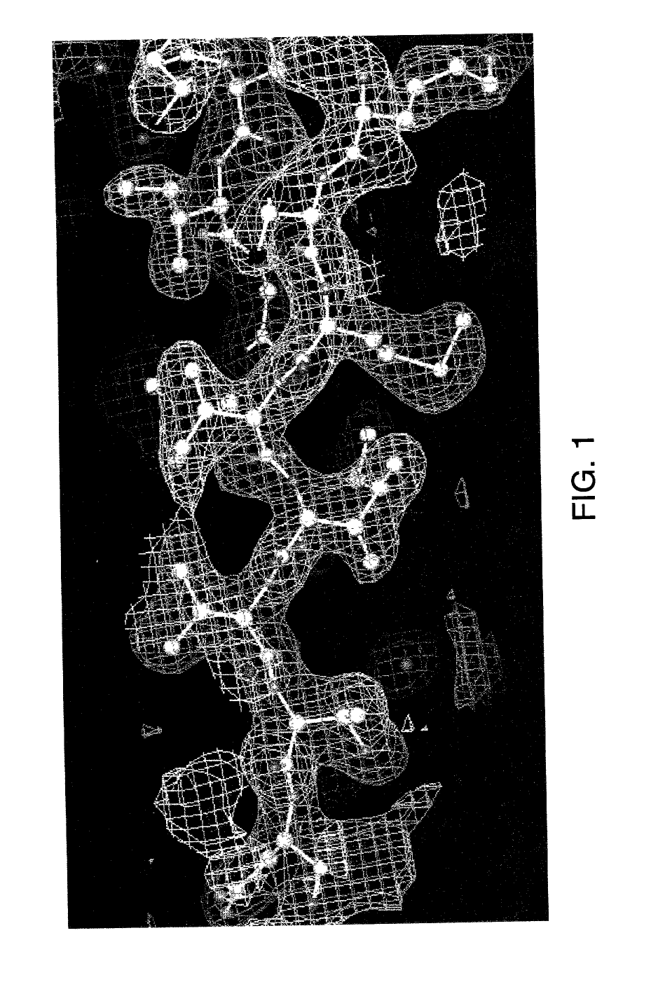

The claimed invention provides crystals of a fragment from the α1 chain of α1β1 integrin(143-340) having unit cells which are rhombohedral, and having the following dimensions a=34.77 Å; b=85.92 Å and c=132.56 Å; α=β=γ=90 Å. Almost all of the residues of the I-domain of the α1 chain of α1β1 integrin, except for residues 143-144 of the N terminus and 336-340 of the C-terminus, are well defined in the final electron density map shown in FIG. 1. The current model consists of 386 amino acid residues and 199 water molecules with a crystallographic R factor of 23.5% and an R_{free} of 30.2% for data between 100 Å and 2.2 Å.

There are two copies of the molecule (termed “A” and “B” ) in the asymmetric unit. The Ramachandran diagram shows that 384 out of the 386 amino acid residues have (Φ,ψ) angles within the allowed regions. The exception is residue Glu192 (A & B). In the atomic coordinates of the rat I-domain crystal structure (Tabld II), residues Thr145, Gln146, Arg234 of molecule A and Thr145 and Arg175 of molecule B are modeled as alanines because of absence of electron density for the side chain. In addition, residues 143, 145, 337, 338, 339,340 of molecule A and 143, 144, 339, 340 of molecule B are not included in the model due to weak electron density.

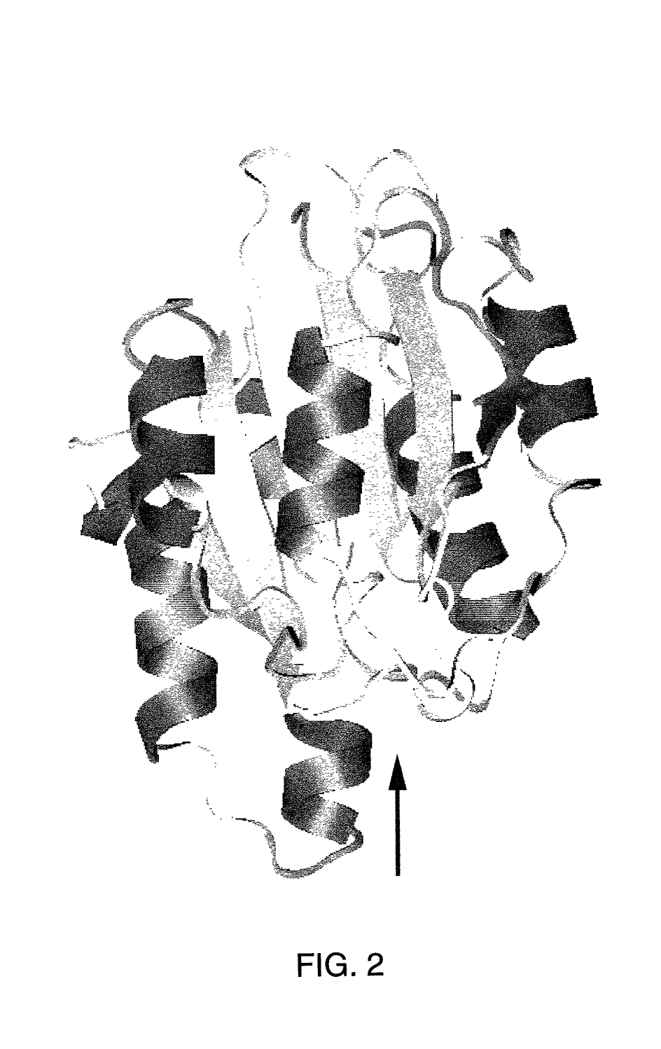

The I-domain adopts the nucleotide-binding fold (FIG. 2) characterized by the existence of seven helices surrounding a core of five parallel β-strands and one antiparallel β-strand. The dimensions of the molecule are 25 Å×30 Å×50 Å. The overall fold is similar to that of αM, αL and α2 I-domains and in particular to that of the α2 I-domain. By homology to the other I-domains it is inferred that the metal-ion-dependent-adhesion-site (MIDAS) of the α1 I-domain consists of residues Asp154, Ser156, Ser158, Thr224, Asp257. The MIDAS site is the site of Mg or Mn cation binding and is expected to be involved in ligand binding. The crystals were grown in the absence of Mg or Mn cations (except for contaminants) and there is no electron density visible in that would correspond to a cation. The structure appears to have the “inactive” conformation according to the model proposed in Lee et al. (1995) Structure 3, 1333-1340. The conformations of molecules A and B are very similar.

(b) Binding Sites

Modeling studies done for collagen binding on the α2 I-domain (Emsley et al. (1997) J. Biol. Chem. 272, 28512-28517) suggest that the binding site for collagen is expected to include the MIDAS site as well as several neighboring residues. By analogy, the binding site of the α1 I-domain for collagen is expected to include residues Asp154, Ser156, Asn157, Ser158, Leu222, Gln223, Thr224, Asp257, Glu259, His261, His288, Tyr289, Gly292, Leu294 and Lys298. Of interest is the observation that the MIDAS site of the α1 I-domain (molecule A in the crystal) forms an interaction with Arg246 of molecule B. It is possible that the positive charge of the arginine side chain replaces the positive charge of the missing metal ion.

(c) Methods of Making an α1β1 Crystal

In various embodiments, the claimed invention relates to methods of preparing crystalline forms of α1β1, or fragments thereof by first providing an aqueous solution comprising α1β1 or a fragment of α1β1. A reservoir solution comprising a precipitating agent is then mixed with a volume of the α1β1 solution and the resultant mixed volume is then crystallized. In certain embodiments, the crystal is derived from an aqueous solution comprising sα1β1(127-340). In preferred embodiments, the crystal is derived from an aqueous solution comprising sα1β1(143-340). The concentration of α1β1 or fragment in the aqueous solution may vary, and is preferably about 1 to about 50 mg/ml, more preferably about 5 mg/ml to about 15 mg/ml, and most preferably, about 10 mg/ml. Similarly, precipitating agents used in the invention may vary, and may be selected from any precipitating agent known in the art. Preferably the precipitating agent is selected from the group consisting of sodium citrate, ammonium sulfate and polyethylene glycol, with polyethylene glycol 8000 being most preferred. Any concentration of precipitating agent may be used in the reservoir solution, however it is preferred that the concentration be about 20% w/v to about 35%w/v, more preferably about 25% w/v. The pH of the reservoir solution may also be varied, preferably between about 4 to about 10, most preferably about 6.5. One skilled in the art will understand that each of these parameters can be varied without undue experimentation and acceptable crystals will still be obtained. In practice, once the appropriate precipitating agents, buffers or other experimental variables are determined for any given growth method, any of these methods or any other methods can be used to grow the claimed crystals. One skilled in the art can determine the variables depending upon his particular needs.

Various methods of crystallization can be used in the claimed invention, including, but not limited to, vapor diffusion, batch, liquid bridge, or dialysis. Vapor diffusion crystallization is preferred. See, e.g. McPherson et al., “Preparation and Analysis of Protein Crystals”, Glick,. Ed., pp 82-159, John Wiley & Co. (1982); Jancarik et.al., “Sparse matrix sampling: a screening method for crystallization of protein”, J. Appl. Cryst. 24, 409-411 (1991), specifically incorporated by reference herein.

In vapor diffusion crystallization, a small volume (i.e. a few milliliters) of protein solution is mixed with a solution containing a precipitating agent. This mixed volume is suspended over a well containing a small amount, i.e. about 1 ml, of precipitating solution. Vapor diffusion from the drop to the well will result in crystal formation in the drop.

The dialysis method of crystallization utilizes a semipermeable size exclusion membrane which retains the protein but allows small molecules (i.e. buffers and precipitating agents) to diffuse in and out. In dialysis, rather than concentrating the protein and the precipitating agent by evaporation, the precipitating agent is allowed to slowly diffuse through the membrane and reduce the solubility of the protein while keeping the protein concentration fixed.

The batch methods generally involve the slow addition of a precipitating agent to an aqueous solution of protein until the solution just becomes turbid, at this point the container can be sealed and left undisturbed for a period of time until crystallization occurs.

Thus, applicants intend that the claimed invention encompass any and all methods of crystallization. One skilled in the art can choose any of such methods and vary the parameters such that the chosen method results in the desired crystals.

(d) Use of ALPHA 1 BETA 1 INTEGRIN Crystal and its Coordinates

The claimed crystals, and coordinates describing them, permit the use of molecular design techniques to design, select and synthesize chemical entities and compounds, including inhibitory compounds or agonists capable of binding to, or associating with, the binding site of α1β1 integrin in whole or in part.

One approach enabled by this invention is the use of the structural coordinates defined herein to design chemical entities that bind to or associate with, α1β1 or fragments of α1β1 and alter the physical properties of the compounds in different ways. Thus, properties such as, for example, solubility, affinity, specificity, potency, on/off rates or other binding characteristics may all be altered and/or optimized.

One may design desired chemical entities by probing a crystal of the present invention with a library of different entities to determine optimal sites for interaction between candidate chemical entities and α1β1 or fragments of α1β1. For example, high resolution x-ray diffraction data collected from crystals saturated with solvent allows the determination of where each type of solvent molecule sticks. Small molecules that bind tightly to those sites can then be designed and synthesized and tested for the desired activity. Once the desired activity is obtained, the molecules can be further optimized.

The claimed invention also makes it possible to computationally screen small molecule data bases or computationally design chemical entities or compounds that can bind in whole, or in part, to extracellular matrix proteins or α1β1 or fragments thereof. They may also be used to solve the crystal structure of mutants, co-complexes, or of the crystalline form of any other molecule homologous to, or capable of associating with, at least a portion of α1β1, i.e., the I-domain of the α1 chain.

One method that may be employed for this purpose is molecular replacement. An unknown crystal structure, which may be any unknown structure, such as, for example, another crystal form of α1β1, an α1β1 mutant, or a co-complex with an extracellular matrix protein such as laminin or collagen, or any other unknown crystal of a chemical entity which associates with α1β1 or fragment which is of interest, may be determined using the structural coordinates of this invention, set forth in Table II. Co-complexes with α1β1 or fragments may include, but are not limited to, laminin-α1β1, collagen-α1β1, and “small molecule”-α1β1. This method will provide an accurate structural form for the unknown crystal more quickly and efficiently than attempting to determine such information without the claimed invention. The information obtained can thus be used to optimize potential inhibitors or agonists of α1β1, and more importantly, to design and synthesize novel classes of chemical entities which will affect the relationship between α1β1 and its ligand(s) in the extracellular matrix.

The design of compounds that inhibit or agonize α1β1 according to this invention generally involves consideration of at least two factors. First, the compound must be capable of physically or structurally associating with α1β1 or a fragment thereof. The association may be any physical, structural, or chemical association, such as, for example, covalent or noncovalent bonding, van der Waals interactions, hydrophobic or electrostatic interactions.

Second, the compound must be able to assume a conformation that allows it to associate with α1β1 or fragment thereof. Although not all portions of the compound will necessarily participate in the association with α1β1 or fragment, those non-participating portions may still influence the overall conformation of the molecule. This in turn may have a significant impact on the desirability of the compound. Such conformational requirements include the overall three-dimensional structure and orientation of the chemical entity or compound in relation to all or a portion of the binding site.

The potential inhibitory or binding effect of a chemical compound on α1β1 or fragment may be analyzed prior to its actual synthesis and testing by the use of computer modeling techniques. If the theoretical structure of the given compound suggests insufficient interaction and association between it and α1β1 or its fragment(s), the need for synthesis and testing of the compound is obviated. However, if computer modeling indicates a strong interaction, the molecule may then be synthesized and tested for its ability to bind to α1β1 or fragment thereof. Thus, expensive and time consuming synthesis of inoperative compounds may be avoided.

An inhibitory or other binding compound of α1β1 or fragment may be computationally evaluated and designed by means of a series of steps in which chemical entities or fragments are screened and selected for their ability to associate with the individual binding sites of α1β1.

Thus, one skilled in the art may use one of several methods to screen chemical entities or fragments for their ability to associate with α1β1 and more particularly, with the individual binding sites of the I-domain of the α1 chain of α1β1(143-340). This process may begin by visual inspection of, for example, the binding site on a computer screen based on the coordinates in Table II. Selected fragments or chemical entities may then be positioned in a variety of orientations, or “docked”, within an individual binding pocket of α1β1. Docking may be accomplished using software such as Quanta and Sybyl, followed by energy minimization and molecular dynamics with standard molecular mechanics force fields, such as CHARMM and AMBER.

Specialized computer programs may be of use for selecting interesting fragments or chemical entities. (GRID, available from Oxford University, Oxford, UK; MCSS or CATALYST, available from Molecular Simulations, Burlington, Mass.; AUTODOCK, available from Scripps Research Institute, La Jolla, Calif.; DOCK available from University of California, San Francisco, Calif., XSITE, University College of London, UK.)

Once suitable chemical entities or fragments have been selected, they can be assembled into an inhibitor or agonist. Assembly may be by visual inspection of the relationship of the fragments to each other on the three-dimensional image displayed on a computer screen, in relation to the structural coordinates disclosed herein.

Alternatively, one may design the desired chemical entities “de novo”, experimentally, using either an empty binding site, or optionally including a portion of a molecule with desired activity. Thus, for example, one may use solid phase screening techniques where either α1β1 or a fragment thereof, or candidate chemical entities to be evaluated are attached to a solid phase thereby identifying potential binders for further study or optimization.

Basically, any molecular modeling techniques may be employed in accordance with the invention; these techniques are known, or readily available to those skilled in the art. It will be understood that the methods and compositions disclosed herein can be used to identify, design or characterize not only entities which will associate or bind to α1β1 or fragment thereof, but alternatively to identify, design or characterize entities which, like α1β1, will bind to extracellular matrix proteins, thereby disrupting the α1β1-ECM interaction. The claimed invention is intended to encompass these methods and compositions broadly.

Once a compound has been designed or selected by the above methods, the efficiency with which that compound may bind to α1β1 or fragment thereof may be tested and optimized using computational or experimental evaluation. Various parameters can be optimized depending on the desired result. These include, but are not limited to, specificity, affinity, on/off rates, hydrophobicity, solubility and other characteristics readily identifiable by the skilled artisan. Thus, one may optionally make substitutions, deletions, or insertions in some of the components of the chemical entities in order to improve or modify the binding properties. Generally, initial substitutions are conservative, i.e. the replacement group will have approximately the same size, shape, hydrophobicity and charge as the original component.

The present invention also enables the design of mutants of α1β1 and the solving of their crystal structure. More particularly, the claimed invention enables one skilled in the art to determine the location of binding sites and interfaces, particularly in the I-domain of the α1 chain, thereby identifying desirable sites for mutation.

For example, mutation may be directed to a particular site or combination of sites on the I-domain, by replacing or substituting one or more amino acid residues. Such mutants may have altered binding properties which may or may not be desirable.

The mutants may be prepared by any methods known in the art, such as for example, site directed mutagenesis, deletion or addition, and then tested for any properties of interest. For example, mutants may be screened for an altered charge at a particular pH, tighter binding, better specificity etc.

Additionally, the claimed invention is useful for the optimization of potential small molecule drug candidates. Thus, the claimed crystal structures can be also be used to obtain information about the crystal structures of complexes of the α1β1 integrin and small molecule inhibitors. For example, if the small molecule inhibitor is co-crystallized with α1β1 or a fragment thereof, then the crystal structure of the complex can be solved by molecular replacement using the known coordinates of α1β1 or fragment for the calculation of phases. Such information is useful, for example, for determining the nature of the interaction between the I-domain of α1β1 integrin and the small molecule inhibitor, and thus, may suggest modifications which would improve binding characteristics such as affinity, specificity and kinetics.

### Example 1

### Determination of Crystal Structure of the ALPHA 1 INTEGRIN I-DOMAIN (127-340)

A. Expression and purification of α1 integrin I-domain.

A soluble fragment of the extracellular domain of rat integrin α1β1 α1 chain containing amino acid residues Val127 to the C-terminal residue Ala340 was produced in soluble form and purified as follows: The gene encoding the rat α1β1 I-domain sequence of amino acids Val127-Ala340 of the α1 chain was amplified from full length cDNAs by the polymerase chain reaction (PCR) (PCR CORE Kit; Boehringer Mannheim, GmbH Germany), using rat specific primers (5′-CAGGATCCGTCAGTCCTACATTTCAA-3′[forward][SEQ ID NO:1]; 5′-TCCTCGAGCGCTTCCAAAGCGAATAT-3′[reverse][SEQ ID NO:2].

The resulting PCR amplified products were purified over a PCR select II column (5 prime-3 prime), digested with Bam H1 and Xho 1 restriction enzymes, re-purified over a PCR select II column, and ligated in pGEX4t (Pharmacia), previously digested with Bam H1 and Xho1, dephosphorylated with calf intestinal alkaline phosphatase (New England Biolabs), and gel purified. Ligation products were transformed into competent DH5A E.Coli cells (Gibco BRL) and the resulting amplicillin resistant colonies were screened for the expression of the 45 kDa glutathione S-transferase-I domain fusion protein. The I-domain was expressed as a GST fusion protein with a thrombin cleavage site at the junction of the sequences.

Cells in PBS (1 part of wet cell weight to 4 parts of buffer) were lysed in a Gaulin press and clarified of particulates by centrifugation (14,000×g, 30 min). 650 ml of lysate from 180 g of cell paste was loaded onto a 25 ml glutathione Sepharose 4B column (Pharmacia). The column was washed with 100 ml of PBS and the rat alpha1 integrin I domain-GST fusion protein eluted from the column with 50 mM Tris HCl pH 8.0, 5 mM glutathione (reduced). Five ml fractions were collected and analyzed for total protein by absorbance at 280 nm and for purity by SDS-PAGE. Peak fractions were pooled, aliquoted, and stored at −70 degrees C. A total of 375 mg of the fusion protein (15 mg/ml) at >90% purity was recovered.

For preparation of the purified I-domain, 6 ml of the fusion protein was dialyzed overnight against one liter of 50 mM Tris pH 7.5. The sample was treated with 100 μg of thrombin (a gift of Dr. John Fenton, New York State Department of Health, Albany, N.Y.) for 150 min at room temperature. DTT was added to 2 mM and the sample was loaded onto a 7 ml glutathione Sepharose® 4B column. The flow through from the column was collected as 1.5 ml fractions and the column was further washed with 50 mM Tris HCl pH 7.5, 2 mM DTT buffer. The flow through and wash fractions were analyzed for absorbance at 280 nm. Peak fractions were pooled and loaded onto a 2.4 ml Q Sepharose® FF column (Pharmacia).

The Q-Sepharose column was washed with 2 ml of 50 mM Tris HCl pH 7.5, 2 mM DTT; 2 ml of 50 mM Tris HCl pH 7.5, 10 mM 2-mercaptoethanol; twice with 2ml of 50 mM Tris HCl pH 7.5, 10 mM 2-mercaptoethanol, 25 mM NaCl; and the alpha 1 integrin I domain eluted with 50 mM Tris HCl pH 7.5, 10 mM 2-mercaptoethanol, 75 mM NaCl. Peak fractions were pooled, filtered through a 0.2 μm filter, and stored at 4 degrees C. The final product was >99% pure by SDS-PAGE, eluted as a single peak by size exclusion chromatography on a Superose® 6 column (Pharmacia & Upjohn) consistent with its predicted mass, and by electrospray ionization-mass spectrometry (ESI-MS, Micromass, Quattro-II, Manchester, UK) contained a single ion with mass of 24,868 Da, which agreed with the predicted mass of 24871.2 Da for the rat α1 I-domain sequence plus the GS linker resulting from cleavage at the engineered thrombin cleavage site. From 72 mg of the fusion protein, 24 mg of the purified I-domain was recovered (based on a theoretical extinction coefficient of 0.5 at 280 nm for 1 mg/ml solution of the I-domain).

In preliminary studies, we found that the rat α1 integrin I-domain in this form failed to crystallize under any test condition and, as had been observed for other I domains (R. Liddington, personal communication), that sequences at the N-terminus of the I domain construct were problematic. A simple proteolytic method was developed to convert the purified rat I-domain into a form that could be crystallized.

Briefly, 240 μl of the purified alpha 1 integrin I domain (16 mg/ml) was diluted with 360 μl of 50 mM Tris HCl pH 7.5 and loaded onto a 1.2 ml V8 protease column (Pierce) that had been equilibrated in 50 mM Tris HCl pH 7.5. The I domain solution was left in contact with the resin for 35 min at room temperature and then recovered by washing the column with 50 mM Tris HCl pH 7.5. The I domain was then dialyzed overnight against 10 mM Tris pH 7.5, 10 mM 2-mercaptoethanol and concentrated to 11 mg/ml in a centricon-10 ultrafiltration unit (Amicon). ESI-MS analysis of V8 protease digested product revealed that the product had been converted into a des 1- 18 form, starting at Cys143 in the fusion protein construct.

B. Crystallization

Buffer chemicals were purchased from Fisher (Boston, Mass.). Crystallization condition screenings were done with the Crystal Screen I kit from Hampton Research (Riverside, Calif.). Crystals were grown by the vapor diffusion method of Jancarik & Kim (1991) J. Appl. Crystallogr. 24, 409-411.

In order to find conditions of crystallization, an incomplete factorial screen was set up. In a typical experiment, protein solution was mixed with an equal volume of reservoir solution and a drop of the mixture was suspended under a glass cover slip over the reservoir solution. Crystals were grown out of 25% w/v Polyethylene Glycol (PEG) 8000, 0.1 M sodium cacodylate pH 6.5, 0.2 M sodium acetate reservoir solution. The crystals are shaped as plates, are easy to reproduce and can reach maximum dimensions of almost 0.5 mm on one side. Variation of pH between 6 and 7 did not affect crystal quality.

Those of skill in the art will appreciate that the aforesaid crystallization conditions can be varied. By varying the crystallization conditions, other crystal forms of α1β1 integrin I-domain may be obtained. Such variations may be used alone or in combination, and include: varying final protein concentrations between 5 mg/ml and 35 mg/ml; varying the sα1β1 integrin I-domain to precipitant ratio; varying PEG concentrations between 15% and 35% w/v; varying the molecular weight of polyethylene glycol from 400 to 8000; varying pH ranges between 5.0 and 9.5; varying sodium cacodylate concentrations between 5 and 395 mM; varying sodium acetate concentrations between 5 and 495 mM; varying the concentration or type of detergent; varying the temperature between −5 degrees C. and 30 degrees C.; and crystallizing α1β1 integrin I-domain by batch, liquid bridge, or dialysis method using the above conditions or variations thereof. See McPherson, A.(1982). Preparation and Analysis of Protein Crystals. (Glick, ed.) pp. 82-159, John Wiley & Co., N.Y., specifically incorporated by reference herein.

C. Data Collection and Processing

Crystals were equilibrated gradually in a cryoprotectant solution of 20% glycerol, 25% w/v PEG 8000, 0.1 M sodium cacodylate pH 6.5, 0.2 M sodium acetate, and were mounted on a loop and immediately frozen in a −150 C. liquid nitrogen gas stream. The technique of freezing the crystals essentially immortalizes them and produced a much higher quality data set.

A native X-ray data set up to 3.0 Å resolution was collected from one crystal by using an R-AXIS II image plate detector system (Molecular Structure Corporation, Woodlands, Tex.). A second data set to 2.2 Å resolution was collected later by using a larger crystal. The data were integrated and reduced using the HKL program package (Otwinowski et al (1993) in Data collection and Processing pp 80-86, SERC Daresbury Laboratory, Warrington, UK). The data collection required about 4 days. Data processing suggested an orthorhombic unit cell with approximate cell dimensions a=34.77 Å, b=85.92 c=132.56 and alpha=beta=gamma=90. The space group was identified as P2₁,2₁2₁. The 2.2 Å data set was 91.3% complete and had an R-merge of 5.6%. Calculation of the Matthews volume gives V_{M}=4.22 assuming a molecular weight of 23,000 daltons which suggested that there are 2 molecules in the asymmetric unit.

D. Molecular Replacement

All subsequent molecular replacement computing was done with the program Amore (Navaja et al (1994) Acta Crystallogr. A 50, 157-163) from the CCP4 program package (The SERC (UK) Collaborative Computing Project No 4, Daresbury Laboratory, UK 1979). Molecular graphics manipulations were done with QUANTA (Molecular Simulations, Inc.) and “O” software (Jones et al 1991 Acta Crystallogr. A 47, 110-119). The coordinates of the crystal structure of the human -60 2 I-domain (Emsley et al. (1997) J. Biol. Chem. 272, 28512-28517) was used as a probe for rotation and translation searches using the 3 Å data set.

We used all the coordinates of all atoms, including side chains. The rotation function gave a solution with the highest correlation coefficient (cc) of 9.7. This solution was used for a first translation function which yielded a cc of 24.6 and an R-factor of 48.7%. Using rigid body refinement, these values refined to cc=40.3, R-factor=48.7%. Using this first solution, we took the peaks of the first rotation search and used these for searching the second molecule, keeping our first solution fixed. The translation search yielded a maximum peak with cc=37.3 and an R-factor of 44.8%. Rigid body refinement on these two solutions resulted in cc=56.3 and R-factor=43.3%.

The next highest solution gave: cc=36.6 R-fac=49.9%. By generating symmetry related molecules and displaying them with computer graphics it was found that they packed satisfactorily in the unit. The rotation matrix between the two molecules of the asymmetric unit was determined and one molecule was used for the initial stages of model building.

E. Model Building and Crystallographic Refinement

All subsequent refinement computing was done with the XPLOR program (Brunger et al (1987) Science 235, 458-460). 10% of the data were used for the calculation of R-free. To reduce model bias, partial models were used for map calculation and refinement. The initial partial model, containing a polyalanine chain of the secondary structure elements only, from the a2 I-domain structure, was subjected to conventional positional refinement and grouped B-factor refinement with strict non -crystallographic symmetry constraints.

The R and R-free factors dropped to 32.3% and 39.4% respectively. 3Fo-2Fc maps were used for cycles of model building and refinement. The resolution range used was from 8 to 3 Å. Typically, cycles consisted of model building, positional refinement and B-factor refinement. When the R and R-free reached 26% and 36% respectively, the 3 Å data set did not allow further improvement of the model. The 2.2 Å data set was collected at this point and was used for all subsequent model building and refinement. The R and R-free factors after the initial rigid body refinement at 2.2 Å were 41.3% and 42.2% respectively.

This larger data set allowed use of simulated annealing refinement and torsion angle dynamics refinement. As the phases improved, more atoms were added into the model. Initially, grouped B-factors were assigned for each residue (one for main chain and the one for side chain atoms). Later, non-crystallographic symmetry constraints were removed and individual atomic B-factors where refined for each residue. In addition bulk solvent correction was applied to the data set. Residues and side chains would be incorporated in the model if they were sufficiently well defined in 3Fo-2Fc electron density maps. Only manual structure modifications that resulted in lower R-free after refinement were accepted.

When R and R-free reached 29% and 34.8% respectively, water molecules were added by using the X-solvate utility of QUANTA. Finally, maximum likelihood refinement was used (Adams et al (1997) Pro. Nat. Acad. Sci USA 94, pp. 5018-5023) and resulted in the final structure with R and R-free of 23.5% and 30.2% respectively for data between 100 and 2.2 Å resolution. Table I summarizes information regarding crystallographic data and refinement. Table II lists the atomic coordinates of the I-domain of the α1 chain of the rat α1β1 integrin. The coordinates of the crystal structure of the I-domain may be used in the structure-based design of small molecule inhibitors of α1β1, computational drug design and iterative structure optimization.

a. Computational Drug Design

Small molecule inhibitors can be designed using computational approaches. These approaches are also known as de novo drug design. In brief, the crystal structure coordinates of the α1β1 integrin or fragment(s) thereof are the input for a computer program, such as DOCK. Programs such as DOCK output a list of small molecule structures that are expected to bind to α1β1 or the fragment(s). These molecules can then be screened by biochemical assays for α1β1 binding. Typically, biochemical assays that screen molecules for their ability to bind to α1β1 or a fragment thereof are competition-type assays. In such assays, the molecule is added to the assay solution and the degree of inhibition is measured using conventional methodology.

An example of such an assay is the following: 96 well plates can be coated with collagen IV or collagen I and blocked with 3% Bovine Serum Albumin solution. Solution of al I-domain together with the small molecule under testing are incubated on the coated plates at room temperature for 1 hour and washed in triton buffer. Bound al I-domain is detected with a biotinylated anti-I-domain antibody. Plates are read at OD₄₀₅ on a microplate reader. The amount of bound I-domain is compared with a control experiment with no small molecule present. If it is lower than that of the control experiment that suggests inhibition by the small molecule.

b. Iterative Cycles of Structure Optimization

The crystal structures of complexes formed between α1β1 or a fragment and small molecule inhibitors may be solved. In brief, small molecule inhibitors are typically found using the crystal structure coordinates of a sα1β1 integrin or fragment either by the computational approaches mentioned above or by the screening of small molecule libraries. The small molecule inhibitor is then co-crystallized with α1β1 or a fragment and the crystal structure of the complex is solved by molecular replacement. Molecular replacement requires the coordinates of a sα1β1 or fragment for the calculation of phases. The information collected from these experiments can be used to optimize the structure of small molecule inhibitors by clarifying how small molecules interact with the protein target. This suggests ways of modifying the small molecule to improve its physicochemical properties, such as affinity, specificity, and kinetics with regard to the α1β1 target.

In addition to being necessary for computational drug design and structure optimization, the crystal coordinates described herein are useful for analyzing the α1β1 binding site. Through such analysis, it was determined that a particularly attractive region for drug targeting is in the vicinity of residues Asp154, Ser156, Asn157, Ser158, Leu222, Gln223, Thr224, Asp257, Glu259, His261, His288, Tyr289, Gly292, Leu294 and Lys298. The above observations and hypotheses suggest that this region may contribute significantly to the binding energy of α1β1/ECM interactions, and therefore, is an attractive target for inhibitor design. Site mutations studies can be used in conjunction with the above-described processes to further define the binding site.

It will be apparent to those skilled in the art that various modifications and variations can be made in the methods and compositions of the present invention without departing from the spirit or scope of the invention. Thus, it is intended that the present invention cover the modifications and variations of this invention provided that they come within the scope of the appended claims and their equivalents.

TABLE I
Crystallographic data statistics:

| Symmetry: | P2₁2₁2₁ |
|---|---|
| Unit cell (Å) | a = 34.77, b = 85.92, c = 132.56 |
| No. of crystals: | 1 |
| Resolution (Å) | 2.2 |
| Reflections(unique): | 19,238 |
| R_{merge} | 5.6% |
| Completeness: | 91.3% |
| Completeness(2.2-2.28 Å) | 77.6% |

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A method of preparing a crystal of at least a portion of α1β1 integrin comprising the steps of:
a) providing an aqueous solution comprising at least a portion of α1β1 integrin;
b) providing a reservoir solution comprising a precipitating agent;
c) mixing a volume of said aqueous solution with a volume of said reservoir solution thereby forming a mixed volume; and
d) crystallizing at least a portion of said mixed volume.

2. The method of claim 1 wherein the aqueous solution of said at least a portion of α1β1 integrin provided in step a) has a concentration of α1β1 integrin of about 1 to about 50 mg per ml.

3. The method of claim 2 wherein the aqueous solution has a concentration of α1β1 integrin of about 5 mg per ml to about 15 mg per ml.

4. The method of claim 3 wherein the aqueous solution has a concentration of α1β1 integrin of about 10 mg per ml.

5. The method of claim 1 wherein the precipitating agent is selected from the group consisting of sodium citrate, ammonium sulfate and polyethylene glycol.

6. The method of claim 1 wherein the concentration of the precipitating agent in the reservoir solution is about 15% w/v to about 35% w/v.

7. The method of claim 6 wherein the concentration of precipitating agent is about 25% w/v.

8. The method of claim 1 wherein the pH of the reservoir solution is about 4 to about 10.

9. The method of claim 8 wherein the pH is about 6.5.

10. The method of claim 1 wherein step d) is by vapor diffusion crystallization, batch crystallization, liquid bridge crystallization or dialysis crystallization.

11. The method of claim 1, wherein the at least a portion of α1β1 integrin comprises at least a portion of an α1 chain of α1β1 integrin.

12. The method of claim 11, wherein the portion of the α1 chain includes an I-domain.

13. A crystal formed by a functional fragment of the extracellular domain of α1β1 integrin or a homolog thereof, the crystal having approximately the following cell constants: a=34.77 Å; b=85.92 Å; c=132.56 Å, γ=90 and a space group of P2₁2₁2₁.

14. The crystal of claim 13, wherein the extracellular domain extends from Cys143 to Ala340 of α1β1 integrin.

15. The crystal according to claim 13 described by the structural coordinates identified in Table II.

16. The crystal of α1β1 integrin according to claim 13, or a homolog thereof, wherein said crystal has a binding site comprising amino acids Asp154, Ser156, Asn157, Leu222, Gln223, Thr224, Asp257, Glu259, His261, His288, Tyr289, Gly292, Leu294 and Lys298.

17. A machine readable data storage medium comprising a data storage material encoded with machine readable data which, when read by an appropriate machine, is capable of displaying a three dimensional representation of a crystal of a molecule or molecular complex comprising a fragment of α1β1 integrin having a binding site comprising amino acids Asp154, Ser156, Asn157, Leu222, Gln223, Thr224, Asp257, Glu259, His261, His288, Tyr289, Gly292, Leu294 and Lys298.

18. A method for determining at least a portion of a three dimensional structure of a molecular complex comprising at least a portion of α1β1 integrin, said method comprising the steps of:
a ) determining the structural coordinates of a crystal of the fragment of α1β1 integrin;
b.) calculating phases from the structural coordinates;
c) calculating an electron density map from the phases obtained in step b);
d) determining the structure of at least a portion of the complex based upon said electron density map.

19. The method of claim 18 wherein the structural coordinates used in step a) are (1) substantially the same as those described in Table II or (2) describe substantially the same crystal as the coordinates in Table II.

20. A method for evaluating the ability of a chemical entity to associate with at least a portion of α1β1 integrin or with at least a portion of an α1β1 integrin receptor, or a complex comprising α1β1 integrin, said receptor, or homologs thereof, said method comprising the steps of:
a) employing computational or experimental means to perform a fitting operation between the chemical entity and said at least a portion of α1β1 integrin or receptor or complex thereof, thereby obtaining data related to said association; and
b) analyzing the data obtained in step a) to determine the characteristics of the association between the chemical entity and said at least a portion of α1β1 integrin or receptor or complex.

21. A chemical entity identified by the method of claim 20, wherein said chemical entity is capable of interfering with the in vivo or in vitro association between an extracellular matrix protein and said at least a portion of α1β1 integrin.

22. A chemical entity identified by the method of claim 20, wherein said chemical entity is capable of associating with a binding site on said at least a portion of α1β1 integrin, wherein said binding site comprises amino acids Asp154, Ser156, Asn157, Leu222, Gln223, Thr224, Asp257, Glu259, His261, His288, Tyr289, Gly292, Leu294 and Lys298.

23. A heavy atom derivative of a crystallized form of at least a portion of α1β1 integrin.

24. A heavy atom derivative of the crystal of claim 23.

25. The use of the structural coordinates of at least a portion of α1β1 integrin to solve a crystal form of a mutant, homologue or co-complex of at least a portion of α1β1 integrin by molecular replacement.

26. A method of obtaining information related to association of a chemical entity with a binding site of at least a portion of α1β1 integrin, the method comprising forming a crystal of at least a portion of α1β1 integrin, or a mutant, or homolog or co-complex of said α1β1 integrin.

27. The method of claim 26 wherein the crystal has the structural coordinates described in Table II.

28. A method for identifying, characterizing or designing a chemical entity having a desired association with at least a portion of α1β1 integrin, comprising the step of determining structural coordinates of a crystal whose structural coordinates are substantially the same as the crystal of α1β1 integrin described in Table II.

29. The method of claim 28, further comprising the step of optimizing the binding characteristics of the chemical entity identified, characterized, or designed.

30. The method of claim 28, further comprising the step of determining the orientation of ligands in a binding site of at least a portion of α1β1 integrin.

31. A chemical entity identified or designed according to claim 28.

32. A method of determining a binding interaction between a chemical entity and at least a portion of α1β1 integrin, the method comprising forming at least a portion of an α1β1 integrin crystal and determining its structual coordinates.

33. The method according to claim 32, wherein said at least a portion of α1β1 integrin crystal is the crystal of claim 13.
